(19) United States
(12) Patent Application Publication
(10) Pub. No.: US 2008/0011297 A1
(43) Pub. Date: Jan. 17, 2008

(54) Monitoring physiologic conditions via transtracheal measurement of respiratory parameters

(76) Inventors: Scott Thomas Mazar, Woodbury, MN (US); Stanley Eugene Kluge, Watertown, MN (US); Thomas Mark Suszynski, Independence, MN (US); Lynn Marlo Zwiers, Lino Lakes, MN (US); Scott Robert Tiesma, New Hope, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/478,936

(22) Filed: Jun. 30, 2006

(51) Int. Cl. G01N 29/02; G01N 19/10; A61M 16/00

(52) U.S. Cl. 128/204.18; 128/204.23; 73/23.24; 73/23.27

(57) ABSTRACT

A method of monitoring a physiologic condition includes suspending, via trans-tracheal implantation, a dual pressure sensor for exposure to a bidirectional airflow within the trachea. A respiratory parameter is measured via the dual pressure sensor based on an airflow-induced pressure differential sensed by the dual pressure sensor, and a physiologic condition is determined via the measured respiratory parameter.

### BACKGROUND

Assessing respiratory functions are an integral part of determining and monitoring the health of an animal or a human. One conventional way of monitoring respiratory functions includes placing an endotracheal tube through the mouth and into the trachea for measuring respiratory functions using a sensor located externally of the airway. Accordingly, in this conventional technique, the instrumentation for making the measurement is remote to the location, i.e. the trachea, in which the measured respiratory function takes place.

Conventional monitoring equipment also alters the natural respiratory functions under study. For example, when an endotracheal tube is placed in the trachea, the natural response of tissues within and adjacent the trachea is altered and the tube causes the airflow within the trachea to become less laminar. This altered respiratory functioning also can be caused by inflatable cuffs used to anchor an endotracheal tube within the trachea. Accordingly, while intubating a patient enables a measurement of respiratory functions, the placement of the endotracheal tube within the trachea alters the respiratory functions that are intended to be measured.

In addition, conventional monitoring equipment is bulky and awkward making it unsuitable for long term monitoring and/or ambulatory monitoring of respiratory functions. Accordingly, the study of the effect of certain medical procedures or the effect of administering pharmaceuticals is greatly limited when monitoring respiratory functions with stationary monitoring equipment.

The health industry and its consumers benefit from the most accurate test information about respiratory functions when evaluating various physiologic conditions of a patient or study animal. Conventional techniques of indirect measurement of respiratory functions continue to limit the accuracy of this test information.

### BRIEF DESCRIPTION OF THE DRAWINGS

FIG. 1 illustrates a plan view of a trans-tracheal sensing system and a block diagram of a sensing monitor of the trans-tracheal sensing system, according to an embodiment of the invention.

FIG. 2A is a sectional view of a trans-tracheal sensing mechanism positioned within a trachea, according to an embodiment of the invention.

FIG. 2B is a sectional view of a trans-tracheal sensing mechanism positioned within a trachea, according to an embodiment of the invention.

FIG. 2C is a top plan view of an anchor for a trans-tracheal sensing mechanism, according to an embodiment of the invention.

FIG. 2D is a side sectional view of a method of implanting a trans-tracheal sensor, according to an embodiment of the invention.

FIG. 3 is a sectional view of a dual pressure sensor of a trans-tracheal sensing mechanism, according to an embodiment of the invention.

FIG. 4 is a sectional view of a dual pressure sensor of a trans-tracheal sensing mechanism, according to an embodiment of the invention.

FIG. 5A is a top plan view of a measurement array, according to an embodiment of the present invention.

FIG. 5B is a schematic diagram of a measurement circuit, according to an embodiment of the invention.

FIG. 6 is a side view of a trans-tracheal sensing mechanism, according to an embodiment of the present invention.

FIG. 7 is a sectional view of a dual pressure sensor of a trans-tracheal sensing mechanism, according to an embodiment of the present invention.

FIG. 8 is a sectional view of a dual pressure sensor of a trans-tracheal sensing mechanism, according to an embodiment of the present invention.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following Detailed Description, therefore, is not to be taken in a limiting sense, and the scope of the present invention is defined by the appended claims.

Embodiments of the invention are directed to sensing respiratory parameters within a trachea of a body to monitor a physiologic condition. In one embodiment, a method comprises suspending a dual pressure sensor within a trachea to detect an airflow-induced pressure differential in the trachea associated with inhalation and exhalation and thereby determine a velocity of the airflow through the trachea. By tracking the velocity of the airflow over a period of time, a sensor monitor determines one or more respiratory parameters, such as a tracheal airway (or gas) pressure, a respiratory tidal volume including inspiration and exhalation volumes, as well as flow rates and other respiratory parameters. The placement of the dual pressure sensor directly in the airflow within the trachea, in combination with the structure of the dual pressure sensor, enables highly accurate measurement of these respiratory parameters.

Analyzing patterns and/or values of these respiratory parameters enables assessing various physiologic conditions, such as sleep apnea, chronic obstructive pulmonary disease (COPD), asthma, pain levels, stress, etc. In another aspect, tracking these respiratory parameters enables analyzing or assessing various aspects of lung mechanics. In another aspect, monitoring these respiratory parameters via the trans-tracheal sensing device enables assessing a physiologic response to pharmaceuticals administered to a patient or study animal, or assessing other interventions intended to alter those physiologic conditions. Accordingly, these applications and numerous other applications of monitoring physiologic conditions are produced from tracking respiratory parameters via trans-tracheal sensing.

In addition, trans-tracheal sensing via embodiments of the invention enables measuring respiratory parameters in a minimally invasive manner to provides minimal interference with normal breathing patterns. This arrangement, in turn, produces lower stress on a test subject, thereby enabling highly accurate long term stationary monitoring or ambulatory monitoring to better mimic real life conditions of a test subject. Conventional airway testing environments are relatively high stress, short term conditions that hinder test accuracy. In embodiments of the invention, longer term monitoring and direct access measurements via trans-tracheal implantation also enable capturing a more complete profile of respiratory parameters on a single test subject, thereby producing more useful test data. Conventional airway testing results are typically based indirect measurements using on average data models from several sets of test subjects.

In one embodiment, a dual pressure sensor obtains measurements via a symmetric arrangement of two substantially identical pressure sensors that provide low sensitivity to temperature and a low sensitivity to motion while accurately capturing airflow data for monitoring respiratory parameters.

In one embodiment, the dual pressure sensor is positioned within the airway of the trachea via a support arm anchored relative to a wall of the trachea. In another embodiment, the dual pressure sensor is positioned externally of the trachea with a pressure sensitive target portion positioned within the trachea. A fluid medium extends within a chamber (which also acts as a support arm) between the pressure sensitive target portion and the dual pressure sensor to transmit pressure sensed at the pressure sensitive target portion from within the trachea to the dual pressure sensor located externally of the trachea. This embodiment enables a lower profile insertion through the trachea and minimizes the amount of space that the sensor occupies within the airway of the trachea.

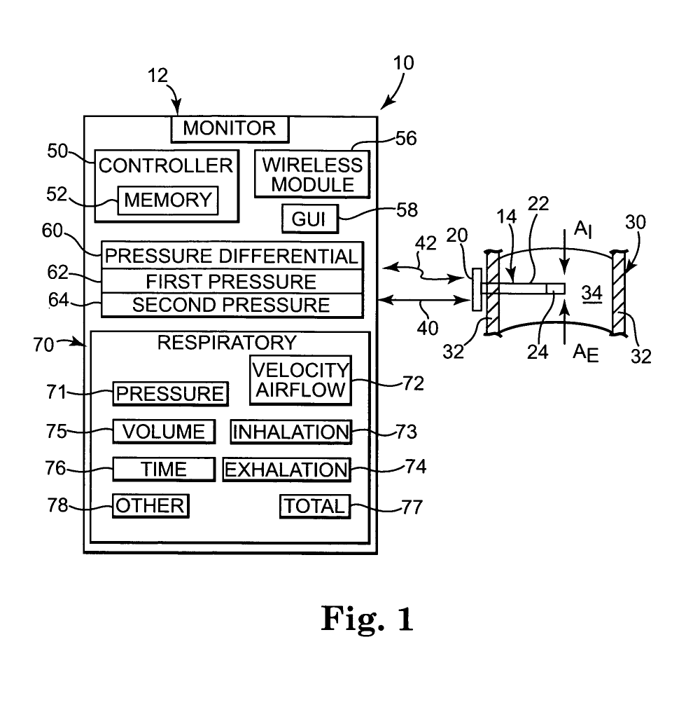

These embodiments and other embodiments of the invention are described and illustrated in association with FIGS. 1-8.

FIG. 1 is a diagram of a trans-tracheal sensing system, according to one embodiment of the invention. As illustrated in FIG. 1, system 10 comprises sensor monitor 12 and trans-tracheal sensor assembly 14 positioned within trachea 30. In one embodiment, sensor assembly 14 comprises flange 20, support arm 22, and dual pressure sensor 24. Trachea 30 comprises wall 32 defining airway 34 for passage of inhalation airflow A_{I} and exhalation airflow A_{E}.

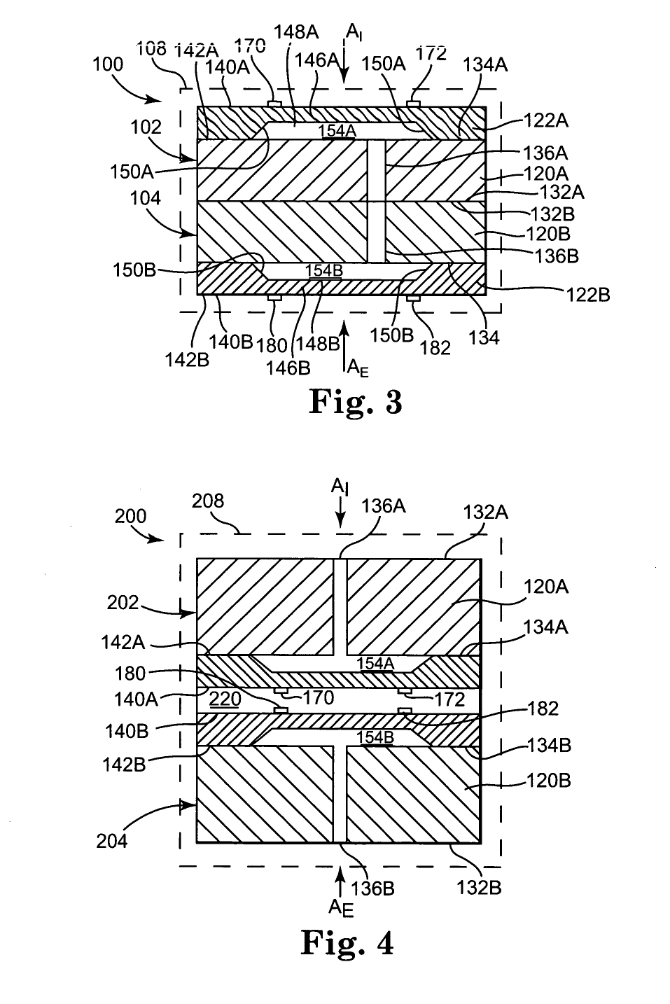

In one aspect, dual pressure sensor 24 of sensor assembly 14 is positioned adjacent an end of support arm 22 opposite from flange 20. Support arm 22 is sized and shaped for slidable insertion through wall 32 of trachea 30 via an insertion tool while flange 20 of sensor assembly 14 is configured to be secured externally relative to wall 32 of trachea 30. In one aspect, support arm 22 has a length sized to extend from flange 20, through wall 32 of trachea 30 to position dual pressure sensor 24 within airway 34 of trachea 30 to enhance accurate measurement of airflows (A_{I} and A_{E}). In one embodiment, dual pressure sensor 24 is positioned adjacent a central axial portion of airway 34 while in other embodiments, dual pressure sensor 24 is positioned in a non-central axial location of airway 34. Additional aspects of dual pressure sensor 24 for accurately measuring respiratory parameters are described and illustrated later in association with FIGS. 3-5B.

In another embodiment, support arm 22 is configured with a length and a generally straight elongate shape to suspend dual pressure sensor 24 in a position within trachea 30 that is generally co-planar relative to support arm 22 and relative to flange 20 located externally of trachea 30. Accordingly, an operator need not direct sensor assembly 14 downward into trachea 30 below the point of trans-tracheal implantation. This arrangement simplifies trans-tracheal implantation of sensor assembly 14 and helps to insure positioning of the dual pressure sensor 24 within airway 34 of trachea 30. In another aspect, support arm 22 forms a resilient, semi-rigid member or a rigid member to facilitate insertion of support arm 22 through wall 32 of trachea 30 and to maintain the position of sensor 24 within trachea 30.

In one aspect, an output signal of dual pressure sensor 24 is communicated via a wired pathway 40 or wireless pathway 42 to sensing monitor 12 for processing to determine various respiratory parameters associated with inhalation and exhalation airflows within trachea 30. In another aspect, wireless communication pathway 42 between sensor assembly 14 and sensing monitor 12 enhances accurate measurements of respiratory parameters because the test subject is no longer tethered to a stationary monitoring station via wired connection, thereby enhancing the freedom of the test subject to behave more naturally during measurement of respiratory parameters.

In one embodiment, sensing monitor 12 of trans-tracheal sensing system 10 comprises controller 50 including memory 52, wireless module 56, and user interface (GUI) 58. Controller 50 controls operation of dual pressure sensor 24, which produces an output signal comprising a pressure differential 60 sensed via dual pressure sensor 24 and which is based on a first pressure 62 associated with a first pressure sensor of dual pressure sensor 24 and a second pressure 64 associated with a second pressure sensor of dual pressure sensor 24.

In one embodiment, sensing monitor 12 determines an array of respiratory parameters based on the pressure differential 60 sensed via pressure sensor 24. Accordingly, sensing monitor 12 also comprises respiratory parameters module 70, which is configured to measure and track a profile of respiratory parameters. In embodiment, respiratory parameter module 70 comprises, but is not limited to, measuring and/or tracking pressure parameter 71, velocity airflow parameter 72, inhalation parameter 73, exhalation parameter 74, volume parameter 75, time parameter 76, total parameter 77, and other respiratory parameter 78. Pressure parameter 71 generally corresponds to an airway pressure within trachea 30 such as an airway pressure during inhalation or exhalation. Velocity airflow parameter 72 comprises a velocity of airflow, which is derived from and proportional to the pressure differential 60 sensed via dual pressure sensor 24. Inhalation parameter 73 generally corresponds to parameters associated with inhalation airflows, such as the velocity airflow during inhalation. Exhalation parameter 74 generally corresponds to parameters associated with exhalation airflows, such as the velocity airflow during exhalation. Volume parameter 75 generally corresponds to volumes derived from an airflow velocity over a time period via time parameter 76, and includes but is not limited to, an inhalation volume, an exhalation volume, or total tidal volume. Total parameter 77 generally corresponds to any respiratory parameter, such as total tidal volume, determined via pressure differential 60 that incorporates both inhalation and exhalation respiratory functions.

Upon determining and tracking any one of respiratory parameters 71-77, one can determine and monitor one or more physiologic conditions about a patient or study animal in which dual pressure sensor 24 is trans-tracheally mounted.

In one embodiment, sensing monitor 12 and/or functions performed by controller 50 of sensing monitor 12 may be implemented in hardware, software, firmware, or any combination thereof. The implementation may be via a microprocessor, programmable logic device, or state machine. Additionally, components of the sensing monitor 12 may reside in software on one or more computer-readable mediums. The term computer-readable medium as used herein is defined to include any kind of memory, volatile or non-volatile, such as floppy disks, hard disks, CD-ROMs, flash memory, read-only memory (ROM), and random access memory.

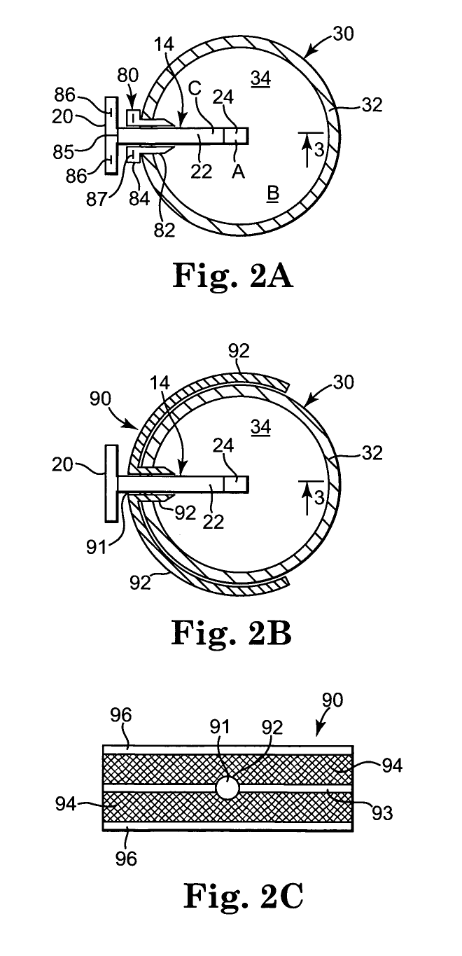

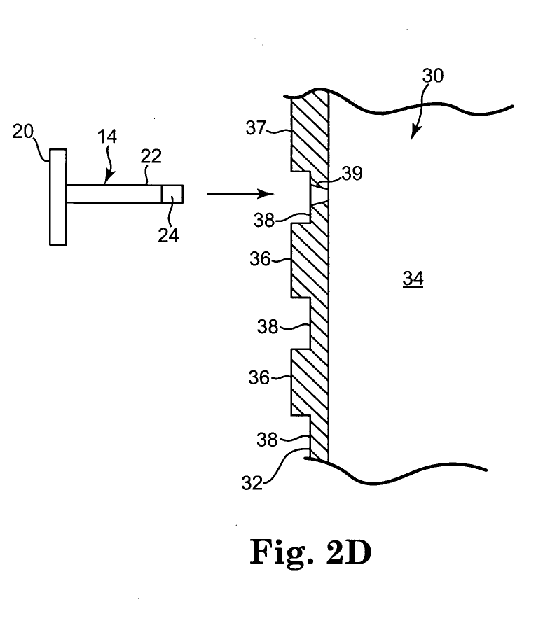

FIG. 2A is sectional view of sensor assembly 14, according to one embodiment of the invention. FIG. 2A illustrates sensor assembly 14 mounted via anchor 80 relative to wall 32 of trachea 30 to suspend dual pressure sensor 24 within airway 34 of trachea 30. In one embodiment, anchor 80 is secured relative to wall 32 of trachea 30 and configured to enable releasable insertion of support arm 22 to support dual pressure sensor 24 within airway 34 of trachea 30. In one aspect, anchor 80 comprises tubular insertion portion 82 and flange 84, with tubular insertion portion 82 sized and shaped for insertion relative to one or more rings of wall 32 of trachea 30. In another aspect, flange 84 is configured for securing anchor 80 relative to an exterior of wall 32 of trachea 30 via suturing, clips, or other securing mechanisms to maintain the position of flange 84 relative to the exterior of wall 32 of trachea 30. Sensor assembly 14 is slidably insertable in tubular portion 82 of anchor 80 to position dual pressure sensor 24 within trachea 30 and for releasable engagement of flange 20 of sensor assembly 14 against flange 84 of anchor 80.

Although FIG. 2A illustrates a small space between flange 20 of sensor assembly 14 and flange 84 of anchor 80 for illustrative clarity, it is understood that upon full slidable insertion of sensor assembly 14 within anchor 80, flange 84 of anchor 80 will in direct contact against flange 20 of sensor assembly 14 to substantially seal the sensor assembly 14 relative to anchor 80 and thereby seal out environmental contaminants and air from entering trachea 30. Additional sealing elements such as viscous fluid, such as lubricant jelly, are used around and on top of mated flanges 84, 20 to further seal out environmental contaminants and keeping body fluids outside of trachea 30. In another aspect, additional sutures, clips, etc. are used to maintain close engagement of flange 20 of sensor assembly 14 relative to flange 84 of anchor 80.

Accordingly, in this arrangement, sensor 24 is suspended within trachea 30 via anchor 80 secured externally of wall 30 of trachea. In addition, in this arrangement, the position of sensor assembly 14 is maintained within airway 34 of trachea 30 while migration of sensor assembly 14 relative to wall 32 of trachea 30 is prevented, thereby insuring robust mounting of sensor assembly 14 during ambulatory monitoring or long-term monitoring.

In another aspect, this arrangement avoids unnecessarily obstructing airway 34 of trachea 30 with structures other than sensor assembly 14 (including support arm 22 and dual pressure sensor 24), thereby generally maintaining the natural inhalation and exhalation airflows through trachea 30. Accordingly, in one embodiment, dual pressure sensor 24 is sized and shaped to have a first surface area A that extends transversely across airway 34 of trachea 30 that is substantially less than a second transverse cross-sectional area B of airway 24 of trachea 30. In one embodiment, the first surface area A of dual pressure sensor 24 occupies about 20% or less of the second transverse cross-sectional area B of trachea 30. In one example of a trachea 30 having a second transverse cross-sectional area B of about 0.8 to 3 cm², the first surface area of dual pressure sensor 24 is about 0.2 cm².

In another aspect, support arm 22 has a third surface area C that extends transversely across airway 34 of trachea 30. Accordingly, in another embodiment, a combination of the first surface area A of dual pressure sensor 24 and the third surface area C of support arm 22 together results in sensor assembly 14 occupying about 20% or less of a second transverse cross-sectional area B of airway 34 of trachea 30. In another embodiment, the combined transverse cross-sectional area of A and C is larger than 20% but presents potential hindrances to natural tracheal functioning and airflow patterns, thereby potentially diminishing accurate measurements of natural respiratory parameters.

In one aspect, dual pressure sensor 24 of sensor assembly 14 is calibrated at the time of its construction to validate its operating characteristics. In one embodiment, to account for the different tracheal diameters for different test subjects, and to the account for the actual position of dual pressure sensor 24 relative to a central portion of airway 34 of the trachea, dual pressure sensor 24 is further calibrated upon its trans-tracheal implantation by comparing measurements at dual pressure sensor 24 with other known indirect measurements of an intra-tracheal pressure via conventional sensing instruments.

In addition, the accuracy of dual pressure sensor 24 and the in-situ calibration of dual pressure sensor 24 also depends, in part, on the alignment of dual pressure sensor 24 to the airflows within trachea 30. Accordingly, in one embodiment, to insure that the pressure sensitive portions of dual pressure sensor 24 are in direct alignment with the airflows to be measured, flange 20 of sensor assembly 14 additionally includes an alignment indicia 85 to facilitate aligning dual pressure sensor 24 within trachea 30. The construction and orientation of these pressure sensitive portions of dual pressure sensor 24 are further described and illustrated in association with FIGS. 3-4.

In another embodiment, a magnetic mechanism releasably secures sensor assembly 14 relative to anchor 80. In particular, as illustrated in FIG. 2A, flange 84 of anchor 80 includes a magnetic component 87 and flange 20 of sensor assembly 14 includes magnetic component 86. With this arrangement, upon slidable insertion of sensor assembly 14 within anchor 80 and slidable mating of the respective flanges 20 and 84, sensor assembly 14 becomes releasably secured relative to the anchor 80 via the interaction of the respective magnetic components 86, 87. In another embodiment, anchor 80 and sensor assembly 14 omits magnetic components 86, 87 and the anchor 80 and sensor assembly 14 are secured relative to one another via other mechanisms.

In one aspect, anchor 90 and sensor assembly 14 (including support arm 22 and dual pressure sensor 24) are made from one or more biocompatible materials and/or are coated with one or more biocompatible coatings, such as parylene, surface treated polyurethane, silicone elastomers, polytetrafluoroethylene, etc. These biocompatible materials and/or coatings maintain the sensitivity and accuracy of dual pressure sensor 24 within a dynamic and harsh biologic environment via maximizing corrosion resistance, promoting shedding of body fluids and contaminants, as well as maximizing surface electrical passivation. Additional embodiments described later in association with FIGS. 2A-8 are constructed of, or coated with, substantially similar materials.

FIG. 2B is a sectional view of a trans-tracheal anchor 90 and sensor assembly 14, according to one embodiment of the invention. As illustrated in FIG. 2B, anchor 90 comprises a generally annular tubular portion 92 and at least one rib 93. The generally tubular portion 92 defines opening 91 to allow slidable insertion of sensor assembly 14. In another aspect, rib 93 defines a generally arcuate shape for extending partially about a circumference of wall 32 of trachea 30. In one aspect, rib 93 stabilizes anchor 90 relative to trachea 30 for implantation, to enable long-term ambulatory monitoring while insuring stable positioning of dual pressure sensor 24 within airway 34 of trachea 30. In substantially the same manner as described for anchor 80 in FIG. 1, anchor 90 provides a mechanism externally of wall 32 of trachea 30 to support dual pressure sensor 24 within airway 34 of trachea 30 without introducing structures other than support arm 22 and dual pressure sensor 24 into airway 34 of trachea 30. In contrast, conventional tracheal pressure monitoring systems typically include an inflatable cuff that occupies a significant portion of trachea 30.

In another embodiment, as illustrated in FIG. 2C, anchor 90 additionally comprises mesh 94 to induce tissue growth onto mesh 94 and rib 93 for securing anchor 90 relative to wall 32 of trachea 30. In one embodiment, anchor 90 additionally comprises outer ribs 96 in addition to central rib 93 to provide additional strength and stability for anchor 90 and to further support mesh 94 relative to anchor 90.

FIG. 2D is a side view illustrating of a method of implanting sensor assembly 14 into and relative to trachea 30, according to an embodiment of the invention. As illustrated in FIG. 2D, trachea 30 comprises wall 32 and airway 34 with wall 32 additionally comprising rings 36 and connective tissue regions 38 (e.g., fibrous tissue, muscle, etc.). These tissue regions 38 are interposed between adjacent rings 36 and connect adjacent rings 36 together into an elongate airway. In one aspect, rings 36 and tissue 38 together define an exterior surface 37 of wall 32 of trachea 30.

Using a puncture tool, an opening 39 is created in wall 32 of trachea 30 to enable insertion and secure implantation of sensor assembly 14 in the manner illustrated in FIGS. 1-2B so that dual pressure sensor 24 is suspended within airway 34 of trachea 30 with flange 20 secured and generally sealed externally relative to wall 32 of trachea 30. In one embodiment, an insertion tool (not shown) is used to puncture an opening 39 in a tissue region 38 between an adjacent pair of rings 36. In one aspect, sensor 24 and support arm 22 are sized and shaped to be slidably insertable through the opening 39 in tissue region 38 between an adjacent pair of rings 36, thereby making this embodiment a minimally invasive implantation procedure. This arrangement avoids cutting through multiple rings 36 of trachea 30.

In another embodiment, a peelable introducer sheath (not shown) is additionally used with the insertion tool to insert sensor 24 and support arm 22 of sensor assembly 14 through wall 32 and into airway 24, whereupon the peelable introducer sheath is removed to leave sensor assembly 14 in place within airway 34 of trachea 30. In one aspect, a dilator is used in conjunction with the peelable introducer sheath to achieve the desired size of opening 39.

In another embodiment, a method of implanting sensor assembly comprises cutting through wall 32 of trachea 30 through one or more rings 36 when necessary to accommodate a larger size sensor assembly 14 or to employ a different surgical technique for securing sensor assembly 14 relative to wall 32 of trachea 30. In this embodiment, opening 39 is larger than that shown in FIG. 2D. Accordingly, sensor assembly 14 is not limited to a size and/or shape for insertion between a pair of adjacent rings 36 of trachea 30, as previously illustrated in association with FIG. 2D.

FIG. 3 is sectional view of a dual pressure sensor 100 for use in trans-tracheal sensing system 10, according to one embodiment of the invention. In one embodiment, dual pressure sensor 100 comprises substantially the same features and attributes as dual pressure sensor 24 as previously described in association with FIGS. 1-2B. In one aspect, dual pressure sensor 100 is positioned at an end of support arm of sensor assembly 14, in a manner substantially the same as dual pressure sensor 24, as illustrated in FIG. 1-2B.

As illustrated in FIG. 3, in one embodiment dual pressure sensor 100 comprises first pressure sensor 102 and second pressure sensor 104 with the respective pressure sensors 102,104 arranged to sense a pressure differential in response to inhalation airflows (AI) and exhalation airflows (AE) within airway 34 of trachea 30 (FIGS. 1-2B). This sensed pressure differential is proportional to a velocity airflow within trachea 30, thereby enabling determination of one or more respiratory parameters via a sensing monitor 12 as previously described and illustrated in association with FIG. 1.

As illustrated in FIG. 3, first pressure sensor 102 comprises base 120A and sensor die 122A including a pressure-sensitive diaphragm portion 146A. In one aspect, base 120A includes a bottom portion 132A, top portion 134A, and inlet 136A. Diaphragm portion 146A of first pressure sensor 102 comprises an exterior top portion 140A, bottom portion 142A, interior portion 148A, and leg portions 150A. A chamber 154A is defined by interior portion 148A and leg portions 150A of diaphragm portion 146A, in combination with top portion 134A of base 120A. Chamber 154 is in fluid communication with air inlet 136A of base 120A.

In one aspect, second pressure sensor 104 comprises substantially the same features and attributes as first pressure sensor 102, with like elements having like reference numerals except being designated as “B” elements. In addition, second pressure sensor 140 is oriented in an opposite direction (i.e., a mirrored relationship) relative to first pressure sensor 102 with the base 120B of second pressure sensor 104 arranged against and secured in contact with base 120A of first pressure sensor 102. This base-to-base arrangement aligns inlet 136A of first pressure sensor 102 to be in fluid communication with inlet 136B of second pressure sensor 104 so that the respective chambers 154A, 154B defined within the respective diaphragm portions 146A, 146B of first and second pressure sensors 102,104 have a common reference pressure and define a closed air volume. This common reference pressure is generally equal to the atmospheric pressure at the time that base 120A of first pressure sensor 102 is connected to and sealed relative to base 120B of second pressure sensor 104.

In addition, the base-to-base arrangement of first and second pressure sensors 102, 104 orients the diaphragm portions 146A, 146B of respective first and second pressure sensors 102,104 to face in opposite directions with first pressure sensor 102 generally facing an inhalation airflow (AI) and second pressure sensor 104 generally facing an exhalation airflow (AE). In this aspect, diaphragm portions 146A extends in a plane that is generally parallel to diaphragm portion 146B. In another aspect, each diaphragm portion 146A, 146B of the respective first and second pressure sensors 102, 104 extends transversely across the airway 34 of the trachea 30 (FIG. 1) to be generally perpendicular to the direction of inhalation airflow A_{I} and/or to the direction of exhalation airflow A_{E} through airway 32 of trachea 30. Accordingly, sensor 100 is positioned on end of support arm 22 of sensor assembly 14, and anchored relative to wall 32 of trachea 30 in a manner to orient diaphragm portions 146A, 146B in a position that is directly responsive to, and therefore the most sensitive to the direction of the inhalation and exhalation airflows (AI, AE). This arrangement enhances the ability to make accurate measurements of respiratory parameters within trachea 30.

In another aspect, diaphragm portion 146A of first pressure sensor 102 is mechanically independent of diaphragm portion 146B of second pressure sensor 104 to insure independent, separate measurements at each respective first and second pressure sensor 102, 104.

In another aspect, establishing a common pressure reference for both first pressure sensor 102 and second pressure sensor 104 (via the sealed base-to-base arrangement) enables dual pressure sensor 100 to sense a pressure differential via diaphragm portions 146A, 146B of the respective first pressure sensor 102 and second pressure sensor 104 based on the exposure of those oppositely oriented diaphragm portions 146A, 146B to the bidirectional airflow in trachea 30. In one aspect, upon an inhalation airflow (AI), a pressure differential is created at sensor 100 with a greater pressure exerted upon diaphragm portion 146A of first pressure sensor 102 (that directly faces the inhalation airflow AI) than upon diaphragm portion 146B of second pressure sensor 104. Likewise, in another aspect, upon an exhalation airflow (AI), a pressure differential is created at sensor 100 with a greater pressure exerted upon diaphragm portion 146B of second pressure sensor 104 (that directly faces the exhalation airflow AE) than upon diaphragm portion 146A of first pressure sensor 102. Accordingly, in one aspect, a direction of airflow is determined by which pressure sensor, either first pressure sensor 102 or second pressure sensor 104 registers the greatest magnitude of pressure.

In another aspect, given that the magnitude of the pressure differential results primarily from either a inhalation providing the dominant pressure signal on the first pressure sensor (with a negligible signal on the second pressure sensor), or from the exhalation providing a dominant pressure signal on the second pressure sensor (with a negligible signal on the first pressure sensor), the pressure differential provides a signal substantially proportional to the airway pressure exhibited during inhalation or during exhalation, respectively.

Sensing monitor 12 processes these pressure signals sensed via dual pressure sensor 100 using a pressure-velocity relationship of Bernoulli's equation in which airflow velocity is proportional to the square root of pressure, with background pressures and gravity effects being negated for this calculation. Accordingly, the pressure differential sensed via dual pressure sensor 100 yields a velocity for either an inhalation airflow (AI) or an exhalation airflow (AE). By tracking the airflow velocity, sensing monitor 12 determines one or more respiratory parameters, such as tidal volumes, airflow rates, etc for either inhalation, exhalation, or both, as previously described and illustrated in association with FIGS. 1-2A. These respiratory parameters, in turn, are used to detect and monitor various physiologic conditions associated with these respiratory parameters.

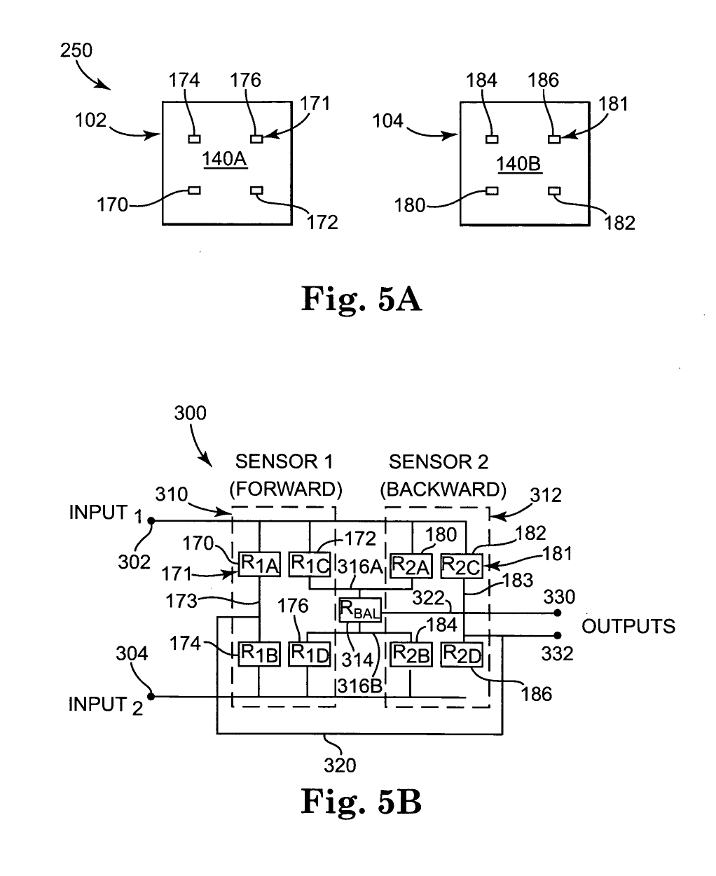

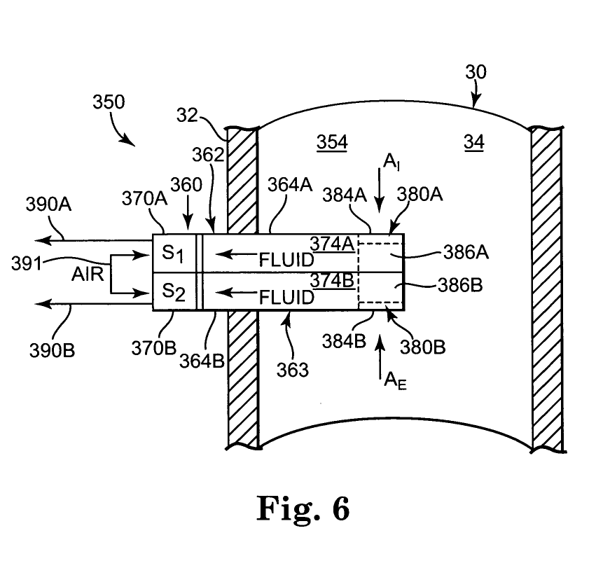

In one aspect, the pressure differential at first pressure sensor 102 and/or second pressure sensor 104 is measured via a sensing circuit 300, as described in more detail later in association with FIGS. 5A-5B. In one aspect, for illustrative purposes, FIG. 3 shows gauges 170, 172 of a first array 171 of gauges 170-178 of sensing circuit 300 and gauges 180, 182 of a second array 181 of gauges 180-188 of sensing circuit 300 as disposed on or incorporated within first and second pressures sensors 102, 104, respectively.

In another aspect, sensor 100 comprises a protective cover 108 that encapsulates first pressure sensor 102 and second pressure sensor 104 to seal out body fluids and other substances that would interfere with the operation of sensors 102, 104. In one aspect, protective cover 108 comprises a thin, flexible and resilient element made of a biocompatible polymer or other material that is resistant to body fluids and other body substances while not interfering with pressure sensing by first and second pressure sensors 102, 104. In one aspect, cover 108 comprises a hydrophobic material or water shedding material to prevent collection of body fluids on cover 108.

FIG. 4 is sectional view of a sensor 200, according to one embodiment of the invention. In one embodiment, sensor 200 comprises substantially the same features and attributes as sensor 100 as previously described in association with FIGS. 1-3, with like reference numerals representing like elements.

In one embodiment, as illustrated in FIG. 4, sensor 200 comprises first pressure sensor 202 and second pressure sensor 204. In one aspect, unlike dual pressure sensor 100, dual pressure sensor 200 comprises a diaphragm portion 146A of first pressure sensor 202 directly faces a diaphragm portion 146B of second pressure sensor 204. By connecting first pressure sensor 202 and second pressure sensor 204 in a face-to-face orientation, an enclosed chamber 220 is interposed between first pressure sensor 202 and second pressure sensor 204. Chamber 220 defines a closed air volume and a common reference pressure for both first pressure sensor 202 and second pressure sensor 204. In a manner substantially similar to the embodiment of FIG. 3, this common pressure reference enables a pressure differential to be sensed by the symmetric pair of sensors 202, 204 at the respective bases 120A, 120B (e.g. via inlets 136A, 136B) of first and second pressure sensors 202, 204.

In one aspect, dual pressure sensor 200 is suspended within airway 34 of trachea 30 (FIG. 1-2A) to orient first pressure sensor 202 and second pressure sensor 204 of dual pressure sensor 200 with their air inlets 136A, 136B (of base 120A, 120B, respectively) in opposite directions within airway 34 so that each air inlet 136A, 136B is aligned substantially directly with a direction of the respective inhalation airflow and exhalation airflow. This arrangement maximizes the impact of the inhalation and exhalation airflows, via air inlets 136A, 136B, on the pressure responsive diaphragm 146A, 146B of each respective first and second pressure sensor 202, 204. As one of the respective inhalation airflow AI or exhalation airflow AE impact sensor 200, a pressure differential is induced between first pressure sensor 202 and second pressure sensor 204 based on the airflow velocity of the respective inhalation and exhalation cycles.

In one aspect, in a manner substantially the same as dual pressure sensor 100, dual pressure sensor 200 senses a pressure differential and a velocity for an inhalation airflow (AI) or exhalation airflow (AE) is determined by sensing monitor 12 (FIG. 1) based on a relationship of airflow velocity and pressure from Bernoulli's Equation. The airflow velocity is then used, via sensing monitor 12, for further determining various respiratory parameters and correlated physiologic conditions.

In one embodiment, dual pressure sensor 200 comprises a cover 208 encapsulating first pressure sensor 202 and second pressure sensor 204 to shield first pressure sensor 202 and second pressure sensor 204 from interference by body fluids within airway 34 of trachea 30.

FIG. 5A is a top plan view of first pressure sensor 102 and second sensor portion 104, according to one embodiment of the invention. As previously introduced in association with FIGS. 3-4, sensing circuit 300 comprises first array 171 of gauges 170-178 and second array 181 of gauges 180-188. In one aspect, FIG. 5A illustrates first array 171 of gauges 170-178 arranged in a generally rectangular pattern on top surface 140A of first pressure sensor 102 and a second array 181 of gauges 180-188 arranged in a generally rectangular pattern on top surface 140B of second pressure sensor 104. Each respective first array 171 of gauges 170-178 and second array 181 of gauges 180-188 are arranged to maximize and accurately sense changes movement in each diaphragm portion 146A, 146B of the respective first and second pressure sensors 102,104 (or of the respective first and second pressure sensors 202, 204) in response to inhalation and exhalation airflows (AI, AE).

FIG. 5B is a schematic diagram of a sensing circuit 300, according to one embodiment of the invention. As illustrated in FIG. 5B, sensing circuit 300 comprises first input 302, second input 304, first output 330, and second output 332. In one aspect, sensing circuit 300 also comprises first sensor portion 310 including first array 171 of gauges 170-78 (as disposed on first pressure sensor 102) for sensing airflow-induced deflections in diaphragm portion 146A of first pressure sensor 102. Second portion 312 of sensing circuit 300 includes second array 181 of gauges 180-188 of second pressure sensor 104 (as disposed on second pressure sensor 204) for sensing airflow-induced deflections in diaphragm portion 146B of first pressure sensor 104.

In one aspect, first sensor portion 310 and second sensor portion 312 are electrically coupled together to produce a differential signal output, which neutralizes noise because of geometrical asymmetry between the first pressure sensor 102 and second pressure sensor 104, as well as neutralizing noise because of as temperature sensitivity, gravitational sensitivity, and other noise characteristics, that are experienced by both first pressure sensor 102 and second pressure sensor 104.

In one aspect, first sensor portion 310 comprises array 171 of gauges represented as resistors 170-178 and second sensor portion 312 comprises array 181 of gauges represented as resistors 180-188, and arranged in a Wheatstone bridge configuration. In one aspect, resistor 172 of first sensor portion 310 is electrically connected to resistor 180 of second sensor portion 312 and resistor 176 of first sensor portion 310 is electrically connected to resistor 184 of second sensor portion 184. In addition, second output 332 is defend by a common node 173, extending between resistor 170 and resistor 174, and by a common node 183, extending between resistor 182 and resistor 186.

In another aspect, a first output 330 of sensing circuit 300 generally corresponds to the output of a balancing resistor 314 (e.g., a potentiometer) that is electrically coupled between common pathways 316A and 316B. Common pathway 316A extends between resistor 172 of first sensor portion 310 and resistor 180 of second sensor portion 312, while common pathway 316B extends between resistor 176 of first sensor portion 310 and resistor 184 of second sensor portion 312. The balancing resistor 314 enables calibrating the output of the respective first and second pressure sensors of a dual pressure sensor, such as first dual pressure sensor 100 (FIG. 3) or second dual pressure sensor 200 (FIG. 4). In particular, adjustments made at balancing resistor 314 enable adjusting a differential signal produced by sensing circuit 300 to counteract noise and/or artifacts common to both the first sensor portion 310 and the second sensor portion 312 while optimizing the interaction of first sensor portion 310 and second sensor portion 312 to insure that accurate detection of a pressure differential at dual pressure sensor 100 or 200, as induced by velocity of inhalation airflow AI and exhalation airflow AE.

FIG. 6 is sectional view of a sensor system 350, according to one embodiment of the invention. As illustrated in FIG. 6, sensor system 350 includes dual pressure sensor assembly 360 that senses a pressure differential associated with an inhalation airflow or an exhalation airflow and provides a corresponding output signal of the sensed pressure differential to a sensing monitor (such as sensing monitor 12 of FIG. 1) for determining various respiratory parameters associated with airflows through trachea 30.

As illustrated in FIG. 6, sensor system 350 includes dual pressure sensor assembly 360 comprising first sensor mechanism 362 and second sensor mechanism 363 arranged in a side-by-side configuration. First sensor mechanism 362 comprises first pressure sensor (S1) 370A, first chamber 364A, and target sensing portion 380A. In one aspect, target sensing portion 380A comprises a pressure sensitive surface 384A and/or a pressure sensitive interior portion 386A. In one aspect, target sensing portion 380A comprises a flexible resilient member capable of deflection in response to air pressure caused by inhalation or exhalation to cause a corresponding movement in sensor portion 370A as transmitted via fluid medium 374A. In one aspect, target sensing portion 380A comprises pressure sensitive surface 384A that directly receives airflow-induced pressure from within trachea 30, which is exerted onto fluid medium 374A. In another aspect, target sensing portion 380B additionally comprises pressure sensitive portion 386A that receives airflow-induced pressure indirectly via pressure sensitive surface 384A, and transmits the pressure to fluid medium 374A. In one embodiment, pressure sensitive portion 384A comprises a gel plug.

In one aspect, chamber 364A of first sensor mechanism 362 is filled with a fluid medium 374A. At one end of chamber 364A, fluid medium 374A is in communication with pressure sensitive portion 384A or 386A and at the other end of chamber 364A, fluid medium 374A is operatively coupled relative to first pressure sensor 370A. In one embodiment, fluid medium 374A comprises a viscous liquid adapted to transmit pressure changes with minimal noise components while in other embodiments, fluid medium 374A comprises air. Accordingly, in one aspect, fluid medium 374A comprises a fluorinert fluid material or similar fluid material.

In another aspect, second sensor mechanism 363 is constructed and operates in a substantially similar manner as first sensor mechanism 362, with like elements designated by like reference numerals except carrying the “B” designation (e.g. fluid medium 374B) instead of the A designation (e.g., fluid medium 374A). However, target sensing portion 380B of second sensor mechanism 363 is oriented in an opposite direction relative to target sensing portion 380A of first sensor mechanism 362. Accordingly, in this arrangement, first sensor mechanism 362 and second sensor mechanism 363 are arranged so that the target sensing portion 380A of first sensor mechanism 362 directly faces an inhalation airflow A_{I} and the target sensing portion 380B of second sensor mechanism 364 directly faces an exhalation airflow (AE).

In one aspect, the first and second pressure sensors 370A, 370B are positioned at one end of the respective first and second sensor mechanisms 362, 363 for location externally of the wall 32 of trachea 30 while target sensing portions 380A, 380B are positioned at an opposite end of the respective sensor mechanisms 362,363 for suspension within the airway 34 of the trachea 30. Accordingly, an inhalation airflow exerted upon target sensing portion 380A is coupled to first pressure sensor 370A via fluid medium 374A and while an airflow exerted upon target sensing portion 380B is coupled to second pressure sensor 370B via fluid medium 374B.

In another aspect, first pressure sensor 370A and second pressure sensor 370B are operatively coupled together via an airway 391 to define a common reference pressure for both first pressure sensor 370A and second pressure sensor 370B, thereby enabling sensing a pressure differential between first sensor mechanism 362 and second sensor mechanism 363.

In one aspect, in a manner substantially the same as dual pressure sensors 100, 200 (of FIGS. 1-5B), dual pressure sensor assembly 360 obtains a pressure differential and via principles of airflow velocity and pressure (via Bernoulli's Equation), a velocity for an inhalation airflow (AI) or exhalation airflow (AE) is determined by sensing monitor 12 (FIG. 1) via pressure signals 390A,390B from respective first and second sensors 370A,370B of dual pressure sensor assembly 360. The airflow velocity is then used, via sensing monitor 12, for further determining various respiratory parameters and correlated physiologic conditions.

In this arrangement, dual pressure sensor assembly 360 provides a low profile trans-tracheal sensing system because the arrangement permits maintaining the relatively larger first and second pressure sensors 370A, 370B externally of wall 32 of trachea 30 while the relatively smaller target sensing portion 380A, 380B are inserted through wall 32 of trachea 30 and suspended within airway 34 of trachea 30. Accordingly, this embodiment enables smaller incisions in trachea 30 and eases design constraints otherwise associated with miniaturizing a full sensor (e.g., dual pressure sensor 100, 200) in order to place the full-size sensor through wall 32 and within airway 34 of trachea 30. For example, in one embodiment, this smaller size arrangement enables inserting the first and second pressure sensing mechanisms 362,363 into the airway 34 of trachea 30 via a very small incision in a tissue region 38 between an adjacent pair of rings 36 of wall 32 of trachea 30.

In addition, the relatively smaller size target sensing portions 380A, 380B and chambers 364A, 364B occupy less space within airway 34 of trachea 30, thereby facilitate accurate measurements because the dual pressure sensor assembly 360 interferes less with the volume and type (e.g., laminar) of flow through airway 34 of trachea 30. For example, in one embodiment, target sensing portions 380A, 380B of dual pressure sensor assembly 360 are sized and shaped to have a first surface area (analogous to first surface area A in FIG. 2A) that extends transversely across airway 34 of trachea 30 that is substantially less than a second transverse cross-sectional area B of airway 24 of trachea 30 (analogous to area B in FIG. 2A). In one embodiment, the first surface area of target sensing portions 380A, 380B is about 20% or less of the second transverse cross-sectional area B of trachea 30.

In another aspect, chambers 364A, 364B of dual pressure sensor assembly 360 define a third surface area C (analogous to C in FIG. 2A) that extends transversely across airway 34 of trachea 30. Accordingly, in another embodiment, a combination of the first surface area A of target sensing portions 380A, 380B and the third surface area C of chambers 364A, 364B results in dual pressure sensor assembly 362 occupying about 20% or less of a second transverse cross-sectional area B of airway 34 of trachea 30. In another embodiment, the combined transverse cross-sectional area of A and C is larger than 20% but presents potentially hindrances to natural tracheal functioning and airflow patterns, thereby potentially diminishing accurate measurements of natural respiratory parameters.

In one embodiment, first and second sensor mechanisms 401 and 403 are arranged to have a length and a generally straight elongate shape to position target sensing portions 380A, 380B within trachea 30 to extend generally co-planar relative to the respective chambers 364A, 364B and relative to the respective pressure sensors 402, 404 located externally of the trachea 30. Accordingly, an operator need not direct sensor assembly 400 downward into trachea 30 below the point of trans-tracheal implantation. This arrangement simplifies trans-tracheal implantation of sensor assembly 400 and helps to insure positioning of the target sensing portions 380A, 380B adjacent a central axial portion of airway 34 of trachea 30.

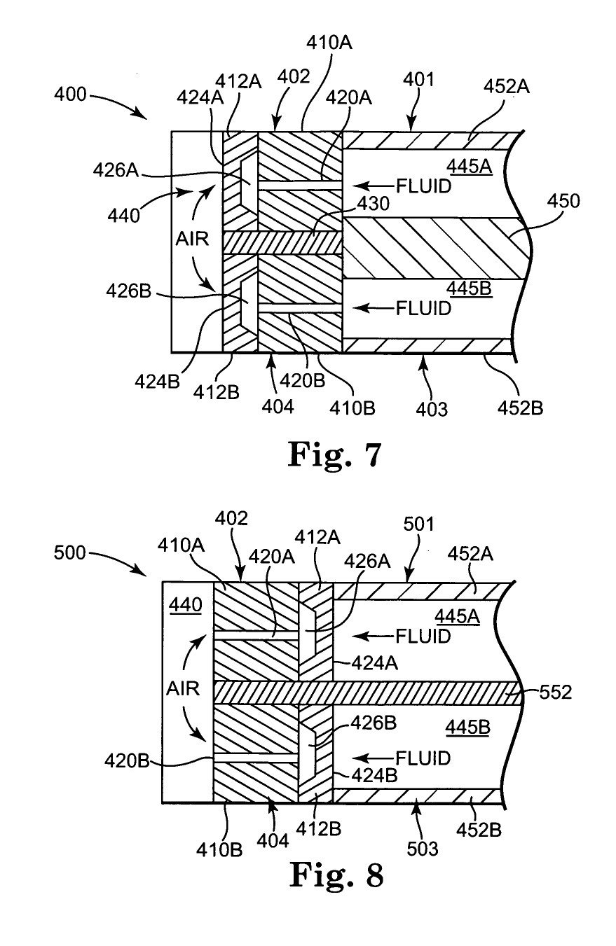

FIG. 7 is sectional view of a dual pressure sensor assembly 400, according to one embodiment of the invention. In one embodiment, dual pressure sensor assembly 400 comprises substantially the same features and attributes as dual pressure sensor assembly 360 as previously described in association with FIG. 6, except with the first and second pressure sensors 370A, 370B of the embodiment of FIG. 7 being replaced by a more specific arrangement of a first pressure sensor 402 and second pressure sensor 404.

In one embodiment, as illustrated in FIG. 7, dual pressure sensor assembly 400 comprises first sensor mechanism 401 and second sensor mechanism 403, which are arranged to sense a pressure differential in response to inhalation airflows (AI) and exhalation airflows (AE) within airway 34 of trachea 30 (FIGS. 1-2B). This sensed pressure differential is proportional to a velocity of inhalation airflow or exhalation airflow within trachea 30, thereby enabling determination of one or more respiratory parameters via a sensing monitor 12 as previously described and illustrated in association with FIG. 1.

As illustrated in FIG. 7, in one embodiment, first sensor mechanism 401 comprises first pressure sensor 402 and a fluid chamber 452A including fluid medium 445A. First pressure sensor 402 comprises base 410A including inlet 420A, sensor die 412A including diaphragm portion 424A, and chamber 426A defined between base 410A and diaphragm portion 424A.

Second sensor mechanism 403 includes second pressure sensor 404 and in all other respects, comprises substantially the same features and attributes as first sensor mechanism 402, with like elements being represented by like reference numerals (except using the B designation instead of the A designation). Accordingly, in one aspect, second sensor mechanism 403 comprise second pressure sensor 404, fluid chamber 452B including fluid medium 445B, and target portion 380B (shown in FIG. 6). In addition, a divider 450 separates fluid chamber 445A and fluid chamber 445B.

As illustrated in FIG. 7, first pressure sensor 402 and second pressure sensor 404 are arranged in a side-by-side configuration with diaphragm portion 424A of first pressure sensor 402 and diaphragm portion 424B of second pressure sensor 404 being exposed to a common reference pressure via a closed volume air chamber (or pathway) 440. As in other embodiments, this common reference pressure provides a common baseline pressure for both first pressure sensor 401 and second pressure sensor 403 to insure accurate sensing of pressure differentials. In one aspect, a divider 430 separates first pressure sensor 402 and second pressure sensor 404, thereby further insuring that first pressure sensor 402 and second pressure sensor 404 operate independently from each other.

In use, an airflow within trachea 30 exerts pressure on target portion 380A,380B (FIG. 6) of the respective first sensor mechanism 401 and second sensor mechanism 403, which is then transmitted via fluid mediums 445A, 445B to the respective first pressure sensor 402 and second pressure sensor 404. In one aspect, each respective fluid medium 445A, 445B is operatively coupled to respective inlets 420A, 420B of first and second pressure sensors 402,404 so that pressure changes within fluid medium 445A, 445B cause a corresponding deflection in diaphragm portions 424A, 424B of first and second pressure sensors 402, 404. The deflections at the respective diaphragm portions 424A, 424B are detected and then produced as a differential signal output, via a sensing circuit 300 as previously described in association with FIGS. 3-5B. Sensing monitor 12 processes this differential signal output to identify an airflow velocity associated with the deflections, and thereby determine the pressure differential associated with a respective inhalation airflow or exhalation airflow.

Accordingly, dual pressure sensor 400 comprises a symmetric arrangement of substantially identical first pressure sensor 402 and second pressure sensor 404, arranged side-by-side, so that differences in pressure sensed via first pressure sensor 402 and second pressure sensor 404 are due substantially to the pressure differential resulting from a simultaneous measurement of an airflow with via two oppositely oriented pressure sensitive elements within an airway of the trachea during inhalation and exhalation airflows.

FIG. 8 is a sectional view of sensor system 500, according to one embodiment of the invention. As illustrated in FIG. 8, sensor system 500 comprises a first sensor mechanism 501 and second sensor mechanism 503 arranged side-by-side in substantially the same manner as respective first sensor mechanism 401 and second sensor mechanism 403 of sensor system 400 of FIG. 7, except with the respective first and second pressure sensors 402 and 404 oriented in an opposite manner relative to fluid chambers 452A, 452B. In one aspect, with first pressure sensor 402 and second pressure sensor 404 arranged in a side-by-side relationship, the diaphragm portions 424A, 424B of the respective first and second pressure sensors 402, 404 are directly coupled relative to the fluid mediums 445A, 445B of sensor system 500. In addition, the inlets 420A, 420B of respective first and second pressure sensors 402, 404 are operatively coupled together via common airway 440 that defines a closed air volume to provide a common reference pressure between first pressure sensor 402 and second pressure sensor 404. In another aspect, a divider 552 separates fluid chamber 452A from fluid chamber 452B and separates first pressure sensor 402 from second pressure sensor 404 to maintain the independence of the operation of first sensor mechanism 501 and second sensor mechanism 503.

In one aspect, an airflow within trachea 30 causes a deflection in pressure sensitive target portions 380A, 380B (FIG. 6), which causes a corresponding pressure change within fluid medium 445A, 445B, which is then transmitted to cause a corresponding deflection in diaphragm portions 424A, 424B of first and second pressure sensors 402, 404. The deflections at the respective diaphragm portions 424A, 424B are detected and then produced as a differential signal output, via a sensing circuit 300 as previously described in association with FIGS. 3-5B. Sensing monitor 12 processes this differential signal output to identify a pressure differential associated with the deflections, and thereby determine the airflow velocity with a respective inhalation airflow or exhalation airflow as well as other respiratory parameters based on the measured airflow velocity.

Embodiments of the invention provide substantially direct and accurate measurements of respiratory parameters associated with inhalation and exhalation airflow within a trachea. These measurements are obtained directly by trans-tracheally suspending a dual pressure sensor within the airway of the trachea or indirectly by trans-tracheally suspending a pressure sensitive target portion within the airway of the trachea and then sensing a pressure change at a dual pressure sensor located externally of the trachea. In either case, a highly accurate measurement of a pressure differential associated with inhalation and exhalation airflows is obtained for use in determining and monitoring various respiratory parameters.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A trans-tracheal sensor device comprising:
a support arm configured for transtracheal implantation and including a first end configured for securing relative to a wall of the trachea and a second end for suspension within an airway of the trachea;
a dual pressure sensing mechanism including a target portion positioned adjacent the second end of the arm, the dual pressure sensing mechanism configured to sense a pressure differential via the target portion in response to a respiratory airflow within the airway of the trachea and configured to produce an output signal for determining a respiratory parameter based on the sensed pressure differential.

2. The trans-tracheal sensor device of claim 1 wherein the support arm is sized and shaped, so that upon securing the arm relative to the wall of the trachea, the support arm extends generally co-planar with the target portion of the dual pressure sensing mechanism.

3. The transtracheal sensor device of claim 1 and further comprising:
a generally tubular anchor configured for transtracheal insertion and securing relative to an exterior portion of the wall of the trachea, the anchor including: (1) a passageway for slidable insertion of the support arm through the anchor; and (2) a flange configured to secure the support arm relative to the anchor.

4. The trans-tracheal sensor device of claim 3 wherein the anchor comprises at least one rib and a mesh portion, the at least one rib configured to extend at least partially about a circumference of the trachea and the mesh portion extending from the at least one rib to induce tissue growth for securing the mesh portion relative to the trachea.

5. The trans-tracheal sensor device of claim 1 wherein the target portion of the pressure sensing mechanism comprises a first target portion and a second target portion that faces in an opposite direction from the first target portion, each respective target portion comprising a pressure sensitive surface and configured to extend generally perpendicular to and across a direction of the respiratory airflow within the airway of the trachea.

6. The trans-tracheal sensor device of claim 5 wherein the pressure sensing mechanism further comprises a first pressure sensor and a second pressure sensor wherein the first pressure sensor is operatively coupled relative to the second pressure sensor via a closed air volume to define a common pressure reference for both respective first and second pressure sensors, wherein each respective first and second pressure sensor includes a diaphragm portion with the diaphragm portion of the respective first and second pressure sensors being mechanically independent of each other.

7. The trans-tracheal sensor device of claim 6 wherein the first target portion generally corresponds to the diaphragm portion of the first pressure sensor and the second target portion corresponds to the diaphragm portion of the second pressure sensor.

8. The trans-tracheal sensor device of claim 6 wherein the first sensor portion is electrically coupled relative to the second sensor portion to produce a differential signal output of the sensed pressure differential.

9. The trans-tracheal sensor device of claim 7 wherein the first pressure sensor and the second pressure sensor are arranged with an exterior portion of the diaphragm portion of the respective pressure sensors facing each other and spaced apart with the closed air volume interposed between the respective exterior portions of the diaphragm portions of the respective first and second pressure sensors.

10. The trans-tracheal sensor device of claim 7 wherein the first pressure sensor and the second pressure sensor comprise a base portion defining an inlet in communication with an interior portion of the diaphragm portion, wherein the base portion of the respective pressure sensors face each other and are connected to each other to align the respective inlets to each other, the inlets and the interior portion of the diaphragm portion of the respective pressure sensors defining the closed air volume.

11. The trans-tracheal sensor device of claim 6 wherein the first pressure sensor is arranged side-by-side relative to the second pressure sensor and the pressure sensing mechanism comprises:
the first pressure sensor operatively coupled to the first target portion via a first fluid medium to transmit pressure at the first target portion to the first pressure sensor; and
the second pressure sensor operatively coupled to the second target portion via a second fluid medium to transmit pressure at the second target portion to the second pressure sensor,
wherein the respective first and second pressure sensors are positioned adjacent the first end of the arm for location externally of the trachea.

12. The trans-tracheal sensor device of claim 11 wherein the first pressure sensor and the second pressure sensor are arranged with an exterior portion of the diaphragm portion of the respective pressure sensors facing the respective first and second fluid mediums.

13. The trans-tracheal sensor device of claim 11 wherein the first pressure sensor and the second pressure sensor include:
the diaphragm portion comprising an interior portion and an exterior portion; and
a base portion connected to the interior portion of the diaphragm portion and defining an inlet in communication with the interior portion of the diaphragm portion,
wherein the inlet of the respective first and second pressure sensors face toward and are operatively coupled relative to the respective first and second fluid mediums, and
wherein the exterior portions of the diaphragm portion of the respective first and second pressure sensors are operatively coupled together via a closed air volume defining a common reference pressure.

14. The trans-tracheal sensor device of claim 1 and further comprising a system including a controller in communication with the sensing mechanism and located remotely from the sensing mechanism to transmit sensed pressure information from the pressure sensing mechanism to the controller, the controller comprising:
a sensing monitor configured to monitor at least one respiratory parameter associated with the sensed pressure information, the at least one respiratory parameter comprising at least one of a respiratory volume, a respiratory flow rate, and a respiratory time period.

15. The trans-tracheal sensor device of claim 14 wherein the controller comprises a communication module in wireless communication with a wireless transceiver of the pressure sensing mechanism.

16. A method of monitoring a physiologic function within a trachea, the method comprising:
suspending, via transtracheal implantation, a dual pressure sensor for exposure to a bidirectional airflow within the trachea; and
measuring a respiratory parameter via the dual pressure sensor based on an airflow-induced pressure differential sensed by the dual pressure sensor; and
determining the physiologic function based on the measured respiratory parameter.

17. The method of claim 16 wherein suspending the at least one sensor comprises:
securing a generally tubular anchor externally relative to a wall of the trachea, the anchor also defining an opening through the outer wall of the trachea;
releasably inserting a first end of a support arm through the anchor to suspend the dual pressure sensor on the first end of the support arm within the trachea; and
releasably securing a second end of the support arm relative to the anchor.

18. The method of claim 16 wherein releasably securing the second end of the support arm comprises magnetically coupling the second end of the support arm relative to the anchor.

19. The method of claim 16 wherein suspending the dual pressure sensor comprises:
arranging the dual pressure sensor to include a first pressure sensor and a second pressure sensor and operatively coupling the first pressure sensor relative to the second pressure sensor via a common air volume, wherein each respective first pressure sensor and the second pressure sensor comprises a diaphragm portion and the diaphragm portion of the first pressure sensor is mechanically independent of the diaphragm portion of the second pressure sensor.

20. The method of claim 19 wherein a measurement circuit of the first sensor portion is electrically coupled relative to a measurement circuit of the second sensor portion to produce a differential signal output of the sensed pressure differential.

21. The method of claim 19 wherein arranging the dual pressure sensor comprises:
orienting an exterior portion of the diaphragm portion of the respective first and second pressure sensors to face toward each other within the airway of the trachea and to be spaced apart from each other with the closed air volume interposed between the respective exterior portions of the first and second sensors; and
arranging the first and second pressure sensor to each include a base portion connected relative to an interior portion of the diaphragm portion, each base portion defining an inlet in fluid communication with interior portion of the diaphragm portion,
wherein the base portions of the respective first and second pressure sensors face in generally opposite directions to expose the inlets relative to the bidirectional airflow within the trachea,
wherein each base portion of the respective first and second pressure sensors extends generally perpendicular to the bidirectional airflow within the trachea.

22. The method of claim 20 wherein arranging the dual pressure sensor comprises:
orienting an exterior portion of the diaphragm portion of the respective first pressure sensor and the second pressure sensor to face away from each other in generally opposite directions within the airway of the trachea, wherein each respective exterior portion of the diaphragm portions extend generally perpendicular to the bidirectional airflow within the trachea;
arranging the first and second pressure sensor to each include a base portion connected relative to an interior portion of the diaphragm portion, each base portion defining an inlet in fluid communication with each other so that the inlets and the interior portions of the diaphragm portion define the common air volume.

23. A method of monitoring a physiologic condition of a body, the method comprising:
transtracheally supporting a pressure sensitive element within an airway of the trachea; and
sensing, via a sensor mechanism located externally of the trachea, a pressure differential adjacent the pressure sensitive element within the airway of the trachea during respiration, the pressure sensitive surface in fluid communication with the sensor mechanism; and
determining a respiratory parameter via the sensed pressure differential and monitoring at least one physiologic condition based on the determined respiratory parameter.

24. The method of claim 23 wherein sensing the pressure differential comprises:
arranging the sensor mechanism to include a first pressure sensor side-by-side relative to a second pressure sensor, and arranging the pressure sensitive element to include a first pressure sensitive element and a second pressure sensitive element facing in an opposite direction relative to the first pressure sensitive element within the airway of the trachea.

25. The method of claim 24 wherein sensing the pressure differential comprises arranging the sensor mechanism to:
operatively couple the first pressure sensor relative to the first pressure sensitive element via a first fluid medium to transmit pressure at the first pressure sensitive element to the first pressure sensor; and
operatively couple the second pressure sensor relative to the second pressure sensitive element via a second fluid medium to transmit pressure at the second pressure sensitive element to the second pressure sensor.

26. The method of claim 24 wherein sensing the pressure differential comprises:
operatively coupling the first pressure sensor and the second pressure sensor together via a closed air volume defining a common reference pressure and with a diaphragm portion of the respective first and second pressure sensors being mechanically independent of each other.

27. The method of claim 24 wherein arranging the sensor mechanism comprises
implementing a strain gauge circuit on each of the respective first and second pressure sensors to sense the pressure differential; and
electrically coupling the strain gauge circuit of the respective first and second pressure sensors to each other to produce a differential signal output representing the sensed pressure differential.
